(19) United States
(12) Patent Application Publication
(10) Pub. No.: US 2006/0269476 A1
(43) Pub. Date: Nov. 30, 2006

(54) Method for integrating large scale biological data with imaging

(76) Inventor: Michael D. Kuo, San Diego, CA (US)

Correspondence Address: Dennis A. Bennett, Esq., 560 Legends View Drive, Eureka, MO, 63025, US

(21) Appl. No.: 11/444,955

(22) Filed: May 31, 2006

Related U.S. Application Data
(60) Provisional application No. 60/685,924, filed on May 31, 2005.

(51) Int. Cl. A61K 51/00

(52) U.S. Cl. 424/1.11

(57) ABSTRACT

There is disclosed a method of extracting large scale biological, biochemical or molecular information about an index disease, biological state, or systems from imaging by correlating the imaging features associated with said disease, state or system with corresponding large scale biological data.

### RELATED APPLICATION(S)

This Application claims priority of U.S. provisional application Ser. No. 60/685,924 filed May 31, 2005 and is incorporated herein by reference.

### FIELD OF THE INVENTION

This invention relates to the field of imaging of patients; more specifically, it relates to using imaging features with corresponding large scale biological data such as gene expression or protein expression data of a patient.

### BACKGROUND OF THE INVENTION

Biomedical imaging is a powerful tool that can provide systems-wide, real time in vivo contextual insights into biology. From the time of the first X-ray, in vivo imaging has provided a vital function for medical research and diagnosis, by permitting the clinician to assess, in real time and space, what is happening within the patient's body. In addition to nuclear medicine and MRI, other imaging methods including positron emission tomography (PET), computerized tomography (CT), ultrasonography (US), optical imaging, infrared imaging, in vivo microscopy and x-ray radiography have also been used for obtaining morphologic, metabolic and functional information of living tissues in vivo in a spatially and temporally resolved manner.

For example, magnetic resonance imaging (MRI) is an imaging technique used primarily in medical settings to produce high quality images of the inside of the body. MRI is based on the absorption and emission of energy in the radio frequency range of the electromagnetic spectrum. Although there is a limitation on imaging objects smaller than the wavelength of the energy being used to image, MRI gets around this limitation by producing images based on spatial variations in the phase and frequency of the radio frequency energy being absorbed and emitted by the imaged object.

Contrast enhanced MRI is a powerful tool for the diagnosis of a variety of malignancies. MRI has both high spatial and temporal resolution, with current imaging systems capable of visualizing changes in tissue contrast with micron spatial resolution and millisecond temporal resolution. It has been demonstrated that malignant tumors tend to have faster and higher levels of enhancement when compared to normal surrounding tissues. Furthermore, the kinetics of contrast enhancement on MRI has been correlated to tumor grades and aggressiveness in different tumors. The precise mechanism and origin of contrast enhancement in tumors therefore seems to be related to the complex biological processes associated with tissue perfusion and vascular permeability such as neovascularization and tumor angiogenesis. This may account for the correlation between tumor grade and aggressiveness and contrast enhancement on MRI.

In the field of nuclear medicine, pathological conditions are localized by imaging the internal distribution of administered radioactively labeled tracer compounds that accumulate specifically at the pathological site. A variety of radionuclides are known to be useful for radioimaging, including .sup.67Ga, .sup.99mTc, .sup.111In .sup.1231, .sup.1251, .sup.169Yb and .sup.186Re. In PET, positron emitting isotopes are conjugated to tracer compounds that also accumulate in pathologic tissues.

Specificity of accumulation may be provided by conjugating the radioactive tracer to a binding moiety that binds to the cells of interest. Many examples of such binding moieties have been used experimentally and clinically. For example, anticancer antibodies labeled with different radionuclides have been studied in human tumor xenografts and in clinical trials. Molecular targets for binding moieties include a variety of tumor-associated antigens. For example, in breast cancer, these molecular targets have included carcinoembryonic antigen (CEA) and the polymorphic epithelial mucin antigen, MUC1, and more recently the growth factor receptors, EGF-R and HER-2/neu. Imaging and image-guided therapeutic agents that target the alpha-v-beta-3 integrin have utilized antibodies conjugated to a liposome surface. Such agents can show changes in spatial and temporal distribution of the receptor using imaging.

Alternatively, radiolabelled peptides have been used for imaging a variety of tumors, infection/inflammation and thrombus. A number of sup.99mTc-label led bioactive peptides and peptidomimetics have proven to be useful diagnostic imaging agents. Due to their small size, these molecules exhibit favorable pharmacokinetic characteristics, such as rapid uptake by target tissue and rapid blood clearance, which potentially allows images to be acquired earlier following the administration of .sup.99mTc-labelled radiopharmaceuticals.

Traditionally, imaging has been used as a noninvasive surrogate for histopathologic assessment of disease and response to treatment. Indeed, the vast majority of advances in biomedical imaging have sought to improve imaging spatial resolution so that imaging can better approach the capabilities of microscopy and histopathology. However, as genomics has demonstrated in recent years, histopathology does not capture much of the underlying molecular diversity inherent in disease processes. It is also clear that the multi-dimensional information provided by clinical imaging is currently underutilized. Presently, the biological detail that imaging can provide is substantially limited because among other things, it relies on the inherent limitations of histopathology, which is the current diagnostic gold standard for discrimination of and characterization of normal and diseased tissue.

Histopathology evaluates the microscopic features of a small section of a tissue (which it then assumes to be representative of the entire tissue) including its composite cells and their surrounding environment and then tries to classify the predominant cell of origin, determine if they are normal or diseased and then subclassify the diseased tissue based on various morphologic features seen by microscopy. However, it is increasingly clear that this type of analysis fails to capture the underlying molecular heterogeneity and diversity that contribute to these disease processes which is evident in histopathology's inability to capture heterogeneous biological processes or predict disease prognosis or treatment outcome with any high level of reliability. Further, pathology relies on tissue for diagnosis and thus is an invasive procedure placing the patient at potential risk any time a histopathologic diagnosis is attempted. But even more, histopathologic analyses are ex vivo representative portraits where the entire disease is assumed to be captured by the snapshot provided by a small representative tissue sampling. Conversely, imaging is a noninvasive tool that can capture in vivo high throughput volumetric data with excellent spatial and temporal resolution. Because it is noninvasive it is inherently safer. Further, imaging can capture real-time, multi-dimensional information about a disease process such as morphologic, physiologic, functional, metabolic, compositional and structural information of an entire system all within the native context of the disease process and against the context of adjacent normal tissues and systems, thus providing global, in vivo and contextual information. DNA microarrays are powerful tools to survey the expression levels of thousands of genes simultaneously. By identifying differential changes in the expression level of many genes simultaneously, thematic expression patterns can emerge that are canonical of underlying biological processes and provide insights into the transcriptional state of a cell. These high throughput biological approaches have been broadly applied to the study of biology including disease and development and have uncovered significant molecular and biologic heterogeneity within a large number of biological systems, processes, states and conditions. For example, in the realm of cancer, these data have permitted delineation of genetic programs and molecular markers associated with tumor biology, treatment response, and prognosis for a large variety of human cancers on a tumor-by-tumor basis.

Further, the recent explosion of information in high throughput biology as exemplified in the fields of genomics, and proteomics has also provided a rich ground for the discovery of molecular targets against which therapeutic and/or diagnostic agents can be directed. Tissues for potential target discovery may include any type of tissue including but not exclusively limited to tumors and other malignant or benign growths, or infected or inflamed tissues. For example, methods have been described for gene expression profiling of tumor cells (see any one of Ono et al. (2000) Cancer Res. 60(18):5007-11; Svaren et al. (2000) J Biol Chem.; or Forozan et al. (2000) Cancer Res. 60(16):4519-25 for examples). Similarly, proteomics has been used to profile the protein expression in tumor samples (see Minowa et al (2000) Electrophoresis 21(9):1782-6; Cole et al. (2000) Electrophoresis 21(9):1772-81; Simpson et al. (2000) Electrophoresis 21(9):1707-32); etc.

While powerful, these genomics approaches currently depend on fresh tissue specimens and specialized equipment. Further, genomic and proteomic analysis is performed on tissue samples without consideration of known differences in imaging patterns within the same tissue over space and time. It would be preferable to acquire gene expression information noninvasively. Further, because current genomics and proteomic approaches still require tissue specimens for analysis, although they can provide much greater molecular detail of a tissue specimen, these approaches still suffer from the same inherent limitations of histopathology as previously described above. Additionally, these current methods of tissue analysis for discovery of new imaging and therapeutic agents do not take into consideration the spatial and temporal variation in gene and protein expression within the target tissues. There is a need to resolve the tissue analysis data both spatially and temporally so that the most relevant targets can be identified. Similarly, there is a clinical need to be able to determine the location and/or extent of sites of focal or localized lesions for initial evaluation, and for following the effects of therapy.

Given this current gap between biomedical imaging, histopathology and new high throughput biological methods, it is evident that new approaches are needed. Clearly, as described above, efforts to make medical imaging a better “noninvasive microscope” suffer from a number of inherent limitations. Conversely, a large number of scientists have tried to resolve these shortcomings with molecular imaging approaches. However, much of the ongoing work in the burgeoning field of molecular imaging focuses on designing new imaging technologies and targeted biologic probes. It is possible however, that many of the imaging characteristics visible using available biomedical imaging modalities reflect molecular properties of underlying states, systems, processes or diseases that are as of yet unrecognized or uncharacterized. Accordingly, it is of interest to determine whether the regulation of gene or protein expression can be correlated with imaging information, thereby allowing imaging to serve as a powerful non-invasive tool for characterizing biological systems, processes, states, conditions, and diseases.

Determining if and how patterns of variation in large scale biological approaches such as genome-wide gene or protein expression data are encoded in dynamic imaging features in biomedical imaging would provide a number of important differential insights. This would allow for example, one to predict strictly based on imaging, regulation of gene or protein expression programs that predict underlying tumor biology, outcome, or response to a particular drug or therapy, and even expression of specific individual genes or proteins of interest. These insights could be used alone or in combination with markers identified from other tests to infer new or differential insights or improve diagnostic accuracy. Similarly, information from this approach could also be used to predict genome wide molecular targets for diagnosis or therapy based on imaging. It is possible that this could all be achieved by the integration of biomedical imaging tools with large scale biological data. This would have far reaching applications for understanding, categorizing and treating disease processes on a molecular level and on a patient-by-patient level.

US 2002/0146371 A1 discloses methods for the discovery, screening and development of novel therapeutic and/or diagnostic targets, based on the use of in vivo imaging of lesions to detect spatial and temporal variations in gene and protein expression. Using the present invention there is provided a broader analysis of gene expression of the index disease as opposed to focusing on particular features than described by the prior art disclosed above. It also allows the analysis without having to obtain a sample from the patient.

### SUMMARY OF THE INVENTION

The present invention is a method of extracting biological information about an index disease, state, condition, system or organism from non-invasive imaging by correlating the imaging features with corresponding large scale biological data. This is achieved generally by providing a specimen having the biological state of interest and collecting or providing large scale biological, biochemical or molecular data of said biological state. The specimen is then imaged and then correlating the information contained in the images of said specimen with the generated or provided large scale biological, biochemical or molecular information to determine an imaging trait that will be indicative of the biological state of interest.

### DETAILED DESCRIPTION

The current invention can be used in many different applications including medical diagnostics, therapeutics, drug discovery and drug testing. Also, given that it is now possible to relate imaging to specific large scale biology and vice versa (relate large scale biology with imaging) this would impact, for example, the design of imaging tools and equipment, imaging protocols, the design, implementation, and interpretation of contrast agents (which are themselves drug-like compounds), software tools for both imaging and the large scale biological data as well as for analyzing and integrating the imaging and genomics, all aspects of drug discovery and testing, patient disease screening, diagnosis and characterization of diseases either by imaging alone or in combination with serological tests. Delineation of the invention and how it in general empowers the aforementioned is detailed below.

The invention comprises correlation of large scale biological data with associated imaging data. Such imaging-large scale biology or imaging-genomic, or radiological-genomic (radiogenomic) analyses yield a detailed and bidirectional association map between the imaging and the associated large-scale biology. The biological data comprises large scale profile data about a particular biological, molecular or biochemical species typically representing a given state. Such data can represent genomic data that might include for example, profiling of gene expression, protein expression or modification, microRNA, DNA copy number, DNA sequence, single nucleotide polymorphisms, or networks, modules or pathways and is characterized by the number of a particular species measured at a given time or state which are greater than one. Examples of large scale data would include but are not limited to gene or protein expression profiling, Serial Analaysis of Gene Expression (SAGE), nuclear magnetic resonance, protein-interaction screens, chromatin immunoprecipitation-Chip, isotope coded affinity tagging, activity based reagents, gel or chromatographic separation, RNAi screens, tissue arrays or mass spectrometry in which a large number of genes, proteins or metabolites are measured in a single experiment or assay.

The imaging data can embody, but is not limited to imaging obtained with magnetic resonance imaging (MRI), nuclear medicine, positron emission tomography (PET), computerized tomography (CT), ultrasonography (US), optical imaging, infrared imaging, in vivo microscopy and x-ray radiography. Imaging can be coupled with medical devices, drugs or compounds, contrast agents or other agents or stimuli that may be used to elicit additional information from the imaging. Images are obtained using these modalities of the lesion, tissue, specimen, system, organism, or patient and can be static or dynamic images both in time and/or space.

The imaging is initially matched to the tissue, specimen, system, organism, or patient from which the large scale biological data is obtained. Imaging information is extracted from each image, imaging study or studies or examinations, and can consists of quantitative or qualitative imaging features that may embody but are not limited to differences in morphology, composition, structure, physiology or function of the lesion, a tissue, specimen, system, organism, or patient. Examples of imaging information include but are not limited to imaging features that may be extracted from multi-phase contrast enhanced dynamic CT, functional imaging, magnetic resonance spectroscopy, diffusion tensor imaging, diffusion or perfusion based imaging as well as targeted imaging encapsulated by nuclear medicine or PET.

The constituent imaging features that are extracted and analyzed as described above, are associated with a given image(s), imaging study(s) or examination(s). These extracted or abstracted image features independently or combinatorally define elements or components of the image, or the composite imaging appearance itself, and are called imaging phenotypes.

The imaging phenotypes are then correlated with the large scale biological data. The resulting imaging phenotype-large scale biological data association is now termed a radiophenotype.

An association map between each radiophenotype and the large scale biological data is thus constructed based on said correlation. The underlying large scale molecular associations with each radiophenotype (and vice versa) are defined as the radiogenotype (i.e. the molecular associations that define, or are associated with a particular radiophenotype(s)). Thus, the association map that is constructed consists of any N number of radiophenotypes associated to any X number of constituents from the large scale biological dataset yielding any Y number of these constituents that are associated to each radiophenotype, resulting in a radiogenotype. These radiophenotype-radiogenotype associations, or radiogenomic associations, result in a detailed association map which can then serve as a reference against which other images, imaging studies or examinations and/or larges scale biology can then be independently and bi-directionally evaluated against. Additionally, new radiophenotypes and radiogenotypes, and thus radiogenomic associations can be constructed and thus defined, from the application of mathematical or logical operations applied to existing associations. An example would be addition or subtraction of radiophenotypes from an existing radiophenotype to create or define a new radiophenotype, or inclusion of conditional statements (e.g. radiophenotype A=radiophenotype X, plus radiophenotype Y and radiophenotype Z, minus radiophenotype 1). Similarly, this can be applied to radiogenotypes to construct new radiogenotypes, or to radiogenomic associations as well. Thus, the radiophenotypes, radiogenotypes, and radiogenomic associations can then all ultimately be evaluated independently of the original association map.

Thus, radiophenotypes are imaging phenotypes that are associated with large scale biology. A radiophenotype, although it is intimately linked to its large scale biological association, can thus, in one embodiment be viewed as a molecular surrogate of its radiogenotype, and can now exist independent of this. Radiogenotypes are the molecular constituents from the large scale biological data that are associated with the radiophenotype. Similarly, radiogenotypes, can in one embodiment, be viewed as surrogates for their underlying imaging phenotype or radiophenotype and can now exist independent of this as well. The bi-directional relationship between each radiophenotype and its radiogenotype is called a radiogenomic association. The association map is the composite of all the radiogenomic associations.

The following examples demonstrate the present invention.

### Example 1

### Identifying Biological Processes At A Molecular Level Using Imaging

Description of the investigation of the ability of bio-medical imaging to non-invasively evaluate contextual genome-wide alterations of an index disease.

In this particular example, the ability of contrast-enhanced magnetic resonance imaging (CE MRI) to systematically evaluate glioblastoma multiforme (GBM) in vivo, on a genome-wide level is described. GBM was chosen as a model disease in this instance because it is the most common and lethal primary malignant brain neoplasm and is characterized by a molecular heterogeneity that is poorly accounted for by both classical diagnostic methods and current clinical outcome predictors. Further, from an imaging perspective, GBM possesses an extremely diverse radiographic appearance on CE MRI which is also the cornerstone for GBM imaging evaluation across nearly every phase of clinical management. Given these factors, it is proposed that aspects of the genomic, and subsequently, components of the previously unaccounted for clinicopathologic diversity of GBM, could be captured by its accompanying and incompletely characterized radiophenotypic diversity to uncover relevant radiogenomic associations.

First described is the general approach. It is reasoned that although there is noise in both imaging and microarray data that their dimensionality is great enough that coordinated and overlapping regions of inherent high signal could be precisely identified with high confidence. Further, it is felt that a reasonable benchmark would be to be able to recapitulate through noninvasive imaging, similar fundamental insights from the companion independent GBM microarray study by Liang et al. Namely, here it is demonstrated that one could (1) identify imaging features or radiophenotypes that reflected fundamental functional gene expression clusters or modules underlying the genomic heterogeneity of GBMs (e.g. cell proliferation, hypoxia and angiogenesis, immune cell etc), and (2) use these radiophenotypes as biomarkers for underlying gene expression clusters that are able to explain some of its previously unaccounted for clinical heterogeneity. Thus, the overall goal in this instance is to construct a relatively simple, yet high precision global GBM association map with sufficient resolution to identify relationships between the imaging appearance, which are captured by particular radiophenotypes, and sets of genes of particular biological interest which are encompassed by their radiogenotypes.

For this study, a group of 22 GBM patients were analyzed, each of which had undergone pre-operative CE MRI of their brain and also had matching GBM cDNA microarray data. In this instance, the large scale biological data (cDNA gene expression data) consisted of analysis of mRNA transcript levels using 2 color cDNA microarrays containing ˜23,000 elements per array representing ˜18,000 unique genes. Next, defined are a set of radiophenotypes against which to analyze and interpret the images. In this instance, radiophenotypes were designed and selected to meet the following general characteristics: (i) to reflect the current armamentarium of GBM radiological evaluation, (ii) to capture the range of intrinsic heterogeneity in the MR imaging appearance of GBM, (iii) to be simple enough to achieve a high measure of consensus as gauged by high inter-observer agreement, and (iv) to take advantage of the multiphasic/multisequence dimensionality that CE-MRI affords. In addition, to meet these objectives several radiophenotypes were developed and modified a priori with the hope of capturing greater radiological guided insight into GBM tumor biology than more commonly used morphological based GBM radiological descriptors. In total, 10 radiophenotypes were selected against which each GBM image was then evaluated (e.g. degree of contrast enhancement, degree of mass effect, tumor to normal adjacent brain transition zone, tumor location etc).

Given this framework, in this particular instance, an approach to determine the relationship between each imaging trait and each clone/gene, and subsequently, each pre-defined GBM gene expression cluster was developed whereby each imaging trait and combination of imaging traits were independently correlated against each of the 2188 well-measured clones in this data set and an individual corrected p-value calculated. It is noted that any number of correlational or statistical methods and approaches can be applied and is independent of the invention itself (e.g. standard correlation, Bayesian networks, ANOVA, T-test, hypergeometric distribution, linear mixed models, Statistical Analysis of Microarrays, Gene Set Enrichment Analysis, VAMPIRE, Cyber T etc.). The corrected individual p values generated from this correlation were then used to generate corrected aggregate p values for each annotated gene expression cluster—radiophenotype pair. Further, other regions with significant radiogenomic associations were identified (beyond the annotated gene expression clusters) to identify other regions of the genome not annotated, but of potential biological interest newly identified by imaging. In the end, a relatively compact composite association map between each radiophenotype and the underlying gene expression clone set was generated.

The global radiogenomic portrait that emerged from this analysis demonstrated striking correlation with the underlying large scale genomic diversity of GBM. Overall, a GBM imaging-genomic map with significant correlation was created which was organized into numerous biological functions. Further, combinations of radiophenotypes added greater specificity, precision and resolution to the association map.

All eight of eight of the annotated GBM gene expression signatures were captured by the evaluated radiophenotypes and with relatively high resolution producing compact radiogenomic associations. Of these 8 gene expression signatures, 7 represented discrete biological processes consisting of groups of genes that were co-regulated and co-expressed and known to share or be involved in the same coherent biological process: hypoxia/angiogenesis, extracellular matrix (ECM), immune, epidermal growth factor receptor (EGFR), glial, neuronal, and cell proliferation. Thus, the association map allowed one to infer activity of specific gene expression programs within a tumor with molecular detail using particular radiophenotypes defined by their radiogenomic associatons and thus could provide insights into real time, in vivo molecular tumor biology on a tumor-by-tumor basis.

### Example 2

### Identifying New Biological Associations Using Imaging

New insights into the function and roles of individual genes as well as groups of genes were identified using this approach as well. For example, a new gene expression program or signature related to cell signaling was uncovered using this method which was found to be associated with and coherently expressed in one particular radiophenotype's radiogenotype. Further, using a network analysis approach, applied to all of the radiophenotypes and 2188 genes, new potential roles or insights to several individual genes and their relationships to other genes through their conjoint or disjoint associations to particular radiophenotypes were uncovered. Such analyses provide new insights into the relationship between the information in large scale biology and the way that it is manifested through imaging as well new raw insights into the roles and functions of biological components in biological systems. It is clear from this description that a similar approach could be readily applied with other types of biological, biochemical or molecular large scale data such as DNA, RNA, protein, network/pathway, or systems data.

### Example 3

### Predicting Patient Prognosis Or Outcome

Patients with the same histopathologic disease diagnosis clearly do not always exhibit the same clinical behavior. In many different cancers for example (brain, breast, lung, prostate etc), patients with the same grade and stage tumor will have wildly divergent outcomes attesting to the fact that current diagnostic measure are unable to dissect much of the clinical heterogeneity within the same disease process. Molecular approaches using large scale biological data have revealed that a large of amount molecular heterogeneity exists even within tumors with the same grade and stage. Further, biological programs, signatures and networks have been identified that are able to reliably segregate patients based on molecular differences into different outcome classes. Applying the approach disclosed in the current invention allows one to similarly dissect patient outcome and prognosis using noninvasive radiophenotypes from the radiogenomic associations that are based on these underlying molecular differences. In the GBM dataset, a radiophenotype was identified that was able to reliably predict patient outcome based on expression of a previously identified underlying gene expression program that was shown to independently predict patient outcome and whose radiogenotype was implicated in neural stem cell biology. Patients with this particular radiophenotype had a survival approximately 2.5 times worse than their counterparts who did not express this radiophenotype. The predictive ability of this radiophenotype as a molecular surrogate was validated in 3 independent datasets. Briefly, MRI images of patients with GBMs were evaluated for the presence or absence of this imaging feature followed by a survival analysis. In all three datasets this radiophenotype, which is molecular surrogate, was able to reliably and accurately segment patients into good and poor prognosis classes demonstrating the predictive power and basis for this new imaging biomarker. Similarly, radiophenotypes that are known to predict an outcome can now be similarly assessed for the molecular basis via radiogenomic associations, and therapies and diagnostics can be appropriately devised against these newly identified targets.

### Example 4

### Predicting Treatment Response

Large scale biological analyses such as functional genomic or sequence analysis approaches have also been used to identify gene expression programs or sequence variation patterns that predict tumor treatment response to particular therapies. By applying the methods embodied by this invention on a primary liver cancer genomics dataset with biphasic contrast enhanced CT imaging, it was shown that radiophenotypes from radiogenomic associations could predict treatment response to a particular drug. In this case, genome-wide gene expression profiles of 30 hepatocellular carcinoma (HCC) tumors were analyzed using DNA microarrays. Each tumor had corresponding dual phase dynamic contrast enhanced imaging. A gene expression program that predicted response to Doxorubicin was evaluated against the evaluated radiophenotypes. A radiophenotype was identified from the association map created that showed strong correlation to the Doxorubicin response gene expression program. Further analysis demonstrated that the radiophenotype was able to segregate out and reliably predict the relative gene expression levels of the constituent genes that were concordant with those that were Doxorubicin sensitive versus those that were Doxorubicin resistant purely based on the radiophenotype. Clearly, a similar approach could be applied to potentially any specific gene, genes or target using the invention. Further, the embodiment would not be limited to drug response but could be broadly applied to predict types of response, on or off target effects, adverse effects, downstream effects on other biological systems etc.

### Example 5

### Correlating With Downstream Large Scale Biological Data

The invention could be applied to multiple different states, tissues, systems, or lesions in order to provide additional or new information and to build increasingly complex radiogenomic models. Diehn et al, using functional genomic approaches, performed genome-wide annotation of subcellular localization of gene expression in a number of different tumors and cell lines. Briefly, he was able to determine both the expression level and subcellular location, on a genome-wide level, of every measured gene. Gene transcripts subcellular location were characterized as either membrane bound, secreted, cytosolic or nuclear. Thus, by adding this dimension it is possible to know not only what genes are differentially expressed, but what subcellular compartments they represent or co-localize to. As these proteins may be shed into different body compartments, such as the serum, cerebrospinal fluid or urine for example, it may be possible to differentially detect their levels in these different compartments to improve diagnosis. This information can be associated directly with the imaging information in a given lesion for example, to characterize both the expression levels associated with a radiophenotype and their subcellular compartmentalization. For example, one could add additional dimensionality to the radiogenotype by characterizing not only what genes are differentially associated with a given radiophenotype, but also the subcellular location of each transcripts with respect to that radiophenotype—i.e. on the cell surface, nucleus, cytosol etc. Such information could be useful in the development of targeted therapies or diagnostics.

Alternatively, downstream large-scale biological information could also be associated indirectly with the imaging information by correlating large scale information from a different body compartment, tissue, lesion, condition or state—such as in the serum or in a different tissue, state or system for example—with the radiophenotypic information of a particular lesion of interest, to determine radiogenomic associations that define relationships between the lesion radiophenotype and expression levels in a downstream or upstream compartment. For example, when a particular radiophenotype is present, the downstream radiogenomic associations, in the serum for example could be inferred, and vice versa. These types of information could also be brought to bear through different types of synergistic associations through their integration to add increasing complexity to the associations. In one application, it is possible to improve diagnostic detection, prediction and accuracy when the invention is used in conjunction with serological profile data; serological profile data in combination with radiogenomic data could be integrated to improve the overall sensitivity, specificity and characterization of a particular disease.

It should be obvious to those skilled in the art that this approach is not limited to the aforementioned body or subcellular compartments described here and is broadly applicable in scope both in terms of the complexity and localization of the different levels of large scale biological data analysis used and their integration with imaging.

### Example 6

### Identifying Diagnostic Or Therapeutic Targets: High Throughput Screening Of Molecular Targets Using Imaging And Large Scale Data

It is clear from the aforementioned descriptions that the invention provides a detailed association map between imaging and large scale biological, biochemical and molecular data. This information can be used to rapidly identify potential diagnostic or therapeutic targets. In one embodiment of the invention, the association map would provide a detailed list of genes or proteins expressed or associated (radiogenotypes) with each particular radiophenotype that is associated with or characteristic of a particular lesion. These radiogenomic associations, in one embodiment, could serve as the basis for the development or use of targeted compounds for detection, diagnosis, characterization or treatment of the lesion. Integration with different types of large scale biological data such as described in example 5 above could further be used, in this example, to further localize the targets as membrane bound or intracellular, or define their functional protein class (e.g. kinases, G-protein etc) for example. This “high throughput” biological screen could then serve as a basis for identifying, screening or developing novel diagnostic or therapeutic compounds, probes, antibodies etc for these targets. Thus “image” based or guided treatments or diagnostics could be readily developed or applied in this embodiment.

### Example 7

### Creating Dynamic or Evolutionary Radiogenomic Associations

Large scale biological or imaging radiogenomic association maps can be created with increasing spatial or temporal diversity to provide differential or evolutionary insights into radiogenomic associations. For example, large scale biological analyses can be acquired and performed in multiple locations based on a given image or images and differences in their radiophenotypic appearance; a tissue can be analyzed in a tumor region that has high perfusion activity and in a region of the tumor that has low perfusion activity, or within the solid portion of the tumor, and in a region of the nonsolid transition zone of the tumor, and differential radiogenomic associations defined. Similarly, radiophenotypes and their radiogenotypes can be defined or re-defined across multiple points in time; a portion of the tumor can be analyzed at time T=0, and then again in the same or a different location at T=3 months, and an association map constructed. Similarly, it would be possible to summate differential changes in the radiophenotypic appearance of a lesion or its radiogenotype over time to create “evolutionary” or “dynamic” radiogenomic association maps. Thus, radiogenomic association maps are not limited to a single lesion, location or time point.

### Example 8

### Radiogenomic Applications And Tools

While population of the radiogenomic database requires an initial basis of large scale biological and imaging data, application of the invention however, ultimately, can become completely independent of this. Each radiogenomic association is ultimately independent and can be decoupled from the association map. The association maps created can be interrogated with simple or complex queries to provide detailed and specific information to an end user in a bidirectional manner whether gleaning for precise biological associations or specific radiophenotypes as detailed in the aforementioned examples. Similarly, imaging, large scale biological and radiogenomic databases can be cross-referenced and integrated to provide increasingly complex and robust reference databases for radiogenomic association maps.

It is also naturally evident from these descriptions that with this invention, imaging equipment, protocols, pulse sequences as well as contrast agents (targeted or nonspecific) can be developed, modified or applied in order to better extract more precise radiogenomic associations or identify new radiogenomic associations. In addition, it is immediately evident that new methods and/or software tools can be defined with the intent of: (1) providing more refined imaging analyses to identify newer or richer radiophenotypes, (2) to extract or define richer correlations or associations against the underlying biology in order to produce more detailed, complex or richer radiogenotypes, (3) to provide more complex, richer or detailed radiogenomic associations between the radiogenotypes and radiophenotypes to provide increasingly more informative or detailed association maps, and (4) user-interfaces and tools that allow users to query, explore, and extract information from points 1-3.

All publications mentioned herein are incorporated herein by reference for the purpose of describing and disclosing, for example, the compounds and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

Those skilled in the art will understand and appreciate that while the present invention has been described with reference to its preferred embodiments and the examples contained herein, certain variations may be made without departing from the scope of the present invention which is limited only by the claims appended hereto. For example, one skilled in the art will understand and appreciate from the foregoing that the methods for making each of the foregoing embodiments differs with each preferred embodiment.

What is claimed:

1. A method of predicting a possible prognosis, treatment outcome, or diagnosis of a patient comprising:
a) imaging said patient;
b) identifying radiophenotype(s) in said images of said patient; and
c) diagnosing or predicting a treatment outcome or prognosis of said patient based on the presence and/or absence of said radiophenotype(s).

2. The method of claim 1 wherein said possible treatment outcome is said patient's possible response to a particular treatment perturbation or drug treatment.

3. The method of claim 2 wherein said treatment outcome is whether said patient will respond to a particular therapy or drug treatment.

4. The method of claim 2 wherein said drug treatment is an adverse response to said therapy or said drug treatment.

5. The method of claim 2 wherein said particular treatment perturbation is a medical device.

6. The method of claim 2 wherein said treatment is an administration of a drug.

7. The method as recited in claim 6 wherein said patient prognosis to said administration of said drug is a desired response.

8. The method as recited in claim 6 wherein said patient prognosis to said administration of said drug is an adverse response.

9. The method of claim 2 wherein said particular treatment perturbation is a focused external energy source.

10. The method of claim 1 wherein said patient prognosis is a clinical, functional, physiological, molecular or biochemical measure of said patient's disease.

11. The method of claim 10 wherein said clinical measure is overall patient survival.

12. The method of claim 10 wherein said measure is disease free survival.

13. The method of claim 10 wherein said measure is likelihood of recurrence.

14. The method of claim 10 wherein said measure is likelihood of metastasis.

15. The method of claim 1 wherein said patient diagnosis is a clinical, functional, physiological, biological, molecular or biochemical description or basis of said patient's disease.

16. The method of claim 15 wherein said diagnosis is a molecular subtype based on RNA expression, function, or modification.

17. The method of claim 15 wherein said diagnosis is a molecular subtype based on microRNA expression, function, or modification.

18. The method of claim 15 wherein said diagnosis is a molecular subtype based on RNAi expression, function, or modification.

19. The method of claim 15 wherein said diagnosis is a molecular subtype based on protein expression, function, or modification.

20. The method of claim 15 wherein said diagnosis is a molecular subtype based on alterations in DNA.

21. The method of claim 20 wherein said alterations in DNA are changes in copy number.

22. The method of claim 20 wherein said alterations in DNA are epigenetic changes

23. The method of claim 15 wherein said diagnosis is a molecular subtype based on cellular pathways, networks or modules or biological systems.

24. The method as recited in claim 1 wherein said biological state is characterized based on said correlation.

25. The method as recited in claim 1 wherein said patient prognosis, treatment outcome, or diagnosis is based on radiophenotypes, radiogenotypes, radiogenomic associations, or an association map.

26. The method as recited in claim 1 wherein said specimen is a tissue, system, organism or animal.

27. The method according to claim 1 wherein said imaging is selected from the group consisting of MRI, MRS, nuclear scintigraphy, PET, CT, ultrasonography, optical imaging, infrared imaging, and x-ray radiography.

28. The method as recited in claim 1 further comprising the use of an imaging agent or image-enhancing agent.

29. The method as recited in claim 1 further comprising the use of an imaging agent having a binding moiety specific for a cell surface molecule that is expressed in said specimen and an image enhancing label.

30. The method of claim 29 wherein said imaging agent is a small molecular weight, extracellular fluid space (ECF) contrast agent.

31. The method according to claim 1 wherein said imaging is repeated throughout the course of a disease of said patient.

32. The method according to claim 1 wherein said patient has a tumor.

33. The method according to claim 29 wherein two or more binding moieties are selected for imaging.

34. The method according to claim 29 further comprising the step of treating said patient with a therapeutic moiety conjugated to said binding moiety.

35. The method according to claim 29 wherein said imaging agent is activated by an enzyme that is expressed in said target tissue and which can induce image enhancement by catalysis.

36. A method of identifying radiophenotype(s) of a biological state of a specimen comprising;
a) Providing large scale biological, biochemical or molecular data of said biological state;
b) Imaging said specimen; and
c) Correlating the information contained in said images with said large scale biological, biochemical or molecular information.

37. The method as recited in claim 36 wherein said large scale biological, biochemical or molecular data of said biological state is created by providing a specimen having said biological state and generating large scale biological, biochemical or molecular data of said biological state of said specimen.

38. The method as recited in claim 36 wherein said specimen is a tissue, system, organism or animal.

39. The method as recited in claim 36 wherein the underlying biological state of said tissue, specimen, system, organism, or patient's is inferred from the structure, function, composition, physiology, morphology or organization of said image of said tissue, specimen, system, organism, or patient.

40. The method as recited in claim 39 wherein said large scale biological data information is at least one of the following; DNA, genes, gene expression, RNA, RNA modifications, epigenetic alterations, proteins, protein modification, protein function, drug sensitivity profiles, drug/chemical toxicity profiles, systemic response profiles, single nuclear polymorphisms (SNPs), Haplotype maps, RNAi screens, microRNA, genome, transcriptome, metabolome, proteome, physiome, phenome, morpheme, interactome, glycome, secretome, ribonome, orfeome, regulome, cellome, operome, unknome, functome, imagome, modulome and motifome and chromosomal, gene mutations, alterations, losses, gains, amplifications, deletions and metabol ites thereof of the above.

41. A method of determining the likelihood of an adverse reaction to a drug candidate in a patient comprising:
a) Determining the pharmacogenomic or pharmacogenetic profile of the drug candidate on a population;
b) Determining adverse responder population to said drug candidate;
c) Image said adverse responder population;
d) imaging said patient;
e) Correlate the image of said adverse responder population to imaging said patient; and
f) Determining whether said patient is a candidate for said drug therapy based on said correlation.

42. A method of determining whether a patient having a disease state is a candidate for a drug therapy comprising:
a) Determining the pharmacogenomic or pharmacogenetic profile of the drug candidate on a population;
b) Determining responder population to said drug candidate;
c) Image said responder population;
d) Imaging said patient;
e) Correlate the image of said responder population to imaging said patient; and
f) Determining whether said patient is a candidate for said drug therapy based on said correlation.

43. A tangible medium of expression comprising data related to radiopheniotype, radiogenotype, radiogenomic association, or association map.

44. The tangible medium of expression of claim 43 which is a database.

45. The tangible medium of expression of claim 43 wherein data are images and/or descriptions of the radiophenotype(s), or radiogenomic association(s).

46. The tangible medium of expression of claim 45 wherein said images and/or descriptions are a reference that is graphical in nature, text, or both.
